(19) United States
(12) Patent Application Publication
(10) Pub. No.: US 2023/0192724 A1
(43) Pub. Date: Jun. 22, 2023

(54) SUNLIGHT-POWERED ROLLING CRYSTALS

(71) Applicant: THE TRUSTEES OF COLUMBIA UNIVERSITY IN THE CITY OF NEW YORK, New York, NY (US)

(72) Inventors: Michael L. Steigerwald, New York, NY (US); Xavier Roy, New York, NY (US); Amymarie K. Bartholomew, New York, NY (US); Ilana B. Stone, New York, NY (US)

(73) Assignee: THE TRUSTEES OF COLUMBIA UNIVERSITY IN THE CITY OF NEW YORK, New York, NY (US)

(21) Appl. No.: 18/145,296

(22) Filed: Dec. 22, 2022

Related U.S. Application Data
(60) Provisional application No. 63/292,775, filed on Dec. 22, 2021.

(51) Int. Cl. C07F 1/08; G03F 7/004

(52) U.S. Cl. CPC C07F 1/08; G03F 7/0042; C07B 2200/13

(57) ABSTRACT

The present subject matter relates to photomechanical compounds and methods for producing such photomechanical materials. The compound can include a solid-state compound having a formula including [Cluster1][Cluster2]. Cluster 1 can include copper and azobenzene, and Cluster 2 can include a counterion.

### CROSS-REFERENCE TO RELATED APPLICATION

This application claims priority to U.S. Provisional Patent Application No. 63/292,775, which was filed on Dec. 22, 2021, the entire contents of which are incorporated by reference herein.

### GRANT INFORMATION

This invention was made with government support under grant numbers DMR-1751949, CBET-2017198, and DMR-2011738 awarded by the National Science Foundation (NSG). The government has certain rights in the invention.

### BACKGROUND

Photoactive materials can be used for microfluidics, information storage, flexible electronics, and artificial muscles. Chromophores that respond to visible light can be desirable as they can avoid the use of harmful ultraviolet (UV) light and be used in devices powered by solar energy.

While certain photoactive materials can photomechanically bend, curl, crack, and slowly crawl under UV light, it can be challenging to respond to visible light in a solid state.

Therefore, there is a need for improved techniques for photoactive solid-state materials that can continuously respond to visible light or sunlight.

### SUMMARY

The disclosed subject matter provides photomechanical compounds and methods for producing such photomechanical materials.

An example compound can include a solid-state compound having a formula: [Cluster1][Cluster2]. In non-limiting embodiments, the Cluster 1 can include copper and azobenzene. Cluster 2 can include a counterion. In non-limiting embodiments, the cluster 1 can include a tetrahedral copper. In non-limiting embodiments, the cluster 1 can include an isocyanoazobenzene. In non-limiting embodiments, the formula can include [Cu(CNAB)₄][PF₆].

In certain embodiments, the compound can roll under a light. In non-limiting embodiments, the light can be a white light. In non-limiting embodiments, the light can be sunlight.

In certain embodiments, the compound can be in a form of a crystal. In non-limiting embodiments, a ratio of a height and a length of the crystal ranges from about 0.02 to about 0.04. In non-limiting embodiments, a ratio of height and width of the crystal is from about 0.61 to about 0.94. In non-limiting embodiments, the crystal can continuously roll at least about 500,000 rotations without damage.

In certain embodiments, a rolling speed can be adjustable based on an irradiance of the light.

In certain embodiments, the compound can further include an oil.

In certain embodiments, the compound can be in a form of a thin film. In non-limiting embodiments, the thin film can be incorporated into a device. In non-limiting embodiments, the device can include a microrobot or a microfluid device.

The disclosed subject matter also provides methods for producing a photomechanical material.

An example method can include synthesizing 4-isocyanoazobenzene (CNAB) from 4-aminoazobenzene; combining the CNAB with a copper salt in acetonitrile ([Cu(acetonitrile)4][PF6]); and forming a [Cu(CNAB)₄][PF₆] as a powder.

In certain embodiments, the method can further include crystallizing the powder by diffusing hexanes into a solution of the [Cu(CNAB)₄][PF₆] in tetrahydrofuran (THF) to produce at least one crystal. In non-limiting embodiments, the crystal can be a rodlike crystal. In non-limiting embodiments, a ratio of height and length of the crystal is from about 0.02 to about 0.04. In non-limiting embodiments, a ratio of height and width of the crystal is from about 0.61 to about 0.94. In certain embodiments, the crystal can continuously roll at least about 500,000 rotations without damage.

The disclosed subject matter will be further described below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A provides a diagram showing the synthesis of [Cu-(CNAB)₄][PF6] in accordance with the disclosed subject matter. FIG. 1B provides a diagram showing an example crystal rolling in accordance with the disclosed subject matter. FIG. 1C provides an image showing an example 1T crystal rolling in accordance with the disclosed subject matter. FIG. 1D provides a graph showing an average rate of rolling on a flat surface vs. white light irradiance for five crystals from one batch of 1T in accordance with the disclosed subject matter.

FIG. 2A provides a diagram showing an example molecular structure of 1T highlighting twisted CNAB L1 in accordance with the disclosed subject matter. FIG. 2B provides a diagram showing an example of congested packing of 1T surrounding L1 in accordance with the disclosed subject matter. FIG. 2C provides a diagram showing an example molecular structure of 1F in accordance with the disclosed subject matter. FIG. 2D provides a diagram showing an example absolute value of the N═N—C—C dihedral angles in 1T (L1—L4) and the two crystallographically disordered CNAB units in 1F (L5 and L5′) in accordance with the disclosed subject matter.

FIG. 3A provides a diagram showing an example CNAB isomerization in accordance with the disclosed subject matter. FIG. 3B provides a graph showing an example electronic absorption spectra of trans- and cis-CNAB and 1 in MeCN as molar absorptivity per CNAB unit in accordance with the disclosed subject matter. FIG. 3C provides a graph showing an example normalized diffuse reflectance solid state electronic absorption spectra of 1T and 1F with DFT-calculated energies and relative oscillator strengths of n-π* transitions of cis- and trans-CNAB for N═N—C—C dihedral angles relevant to the structures of 1T and 1F in accordance with the disclosed subject matter. FIG. 3D provides a graph showing an example normalized diffuse reflectance solid-state electronic absorption spectra of 1T before and after exposure to light in accordance with the disclosed subject matter.

FIG. 4A provides a diagram showing an example wavelength range of 1T bending and straightening in accordance with the disclosed subject matter. FIG. 4B provides images showing an example stepwise breakdown of 1T crystal rolling 180° under white light in accordance with the disclosed subject matter.

FIGS. 5A-5F provide optical microscope images showing that (5A-5C) straight crystals bend under (5A) 450 nm and (5B) 530 nm light but not under (5C) 540 nm light, and (5D-5F) bent crystals straighten under (5E) 560 nm light but not under (5D) 550 nm light or (5F) 570 nm light.

FIG. 6 provides a diagram showing an example solid-state molecular packing of [Cu(CNAB)4][PF6] (1T) in accordance with the disclosed subject matter.

FIG. 7 provides a diagram showing an example Solid state molecular packing of [Cu(CNAB)4][PF6]⋅hexanes (1F) with the disordered hexane channels in accordance with the disclosed subject matter.

FIGS. 8A-8C provide a diagram showing an example (8A) solid-state molecular structure of the asymmetric unit of [Cu(CNAB)4][PF6]⋅hexanes (1F) showing the disordered CNAB fragment with (8B) L5 65% and (8C) L5′ 35% occupancy in accordance with the disclosed subject matter.

FIG. 9 provides a graph showing an average rate of rolling vs. white light irradiance for five crystals from Batch 1 of 1T in accordance with the disclosed subject matter.

FIG. 10 provides a graph showing an example diffuse reflectance solid state electronic absorption spectra of crushed crystals of 1T before and after exposure to light of irradiance in accordance with the disclosed subject matter.

FIG. 11 provides a graph showing an example of diffuse reflectance solid state electronic absorption spectra of crushed crystals of 1T before and after exposure to 4 light and upon thermal relaxation back to the trans in the dark in accordance with the disclosed subject matter.

FIG. 12 provides a graph showing an example of diffuse reflectance solid state electronic absorption spectra of intact crystals of 1T before and after exposure to light and upon thermal relaxation back to the trans in the dark in accordance with the disclosed subject matter.

FIG. 13 provides a graph showing an example normalized diffuse reflectance solid state electronic absorption spectra of intact crystals of 1T before and after exposure to light and upon thermal relaxation back to the trans in the dark in accordance with the disclosed subject matter.

FIG. 14 provides a graph showing an example diffuse reflectance solid state electronic absorption spectra of intact crystals of 1F before and after exposure to light and upon sitting in the dark after the light exposure in accordance with the disclosed subject matter.

FIG. 15 provides a graph showing an example normalized diffuse reflectance solid state electronic absorption spectra of intact crystals of 1F before and after exposure to light and upon sitting in the dark after the light exposure in accordance with the disclosed subject matter.

FIG. 16 provides images showing example crystallographic axes derived from a partial SCXRD dataset collection on a crystal of 1T (top) and optical microscope images of the same crystal before (1) and during (2-5) exposure to white light (bottom) in accordance with the disclosed subject matter.

FIG. 17 provides images showing example crystallographic axes derived from a partial SCXRD dataset collection on a crystal of 1T (top) and optical microscope images of the same crystal before (1) and during (2-4) exposure to white light (bottom) in accordance with the disclosed subject matter.

FIG. 18 provides images showing example crystallographic axes derived from a partial SCXRD dataset collection on a crystal of 1T (top) and optical microscope images of the same crystal before (1) and during (2-4) exposure to white light (bottom) in accordance with the disclosed subject matter.

FIG. 19 provides optical microscope images showing three example crystals of 1F before and after exposure to 456 nm blue light and white light in accordance with the disclosed subject matter.

FIG. 20 provides images showing the disclosed crystal of 1T before and after exposure to white light and as it relaxed back to the unbent state under ambient light at room temperature in accordance with the disclosed subject matter.

FIG. 21 provides optical microscope images showing several crystals of 1T of various dimensions before and after exposure to white light and as they relaxed back to the unbent state under ambient light at room temperature in accordance with the disclosed subject matter.

FIG. 22 provides graphs showing ¹H NMR of all-trans-1 obtained by dissolving a sample of 1T crystals, freezing that solution in liquid nitrogen, and then thawing the solution just before a collection of the ¹H NMR (top), with tentative peak assignments (bottom) in accordance with the disclosed subject matter.

FIG. 23 provides graphs showing 1H NMR spectra collected at various time intervals in accordance with the disclosed subject matter.

FIG. 24 provides graphs showing (Bottom) 1H NMR of 1T crushed crystals, (Middle) 1H NMR of 1T crushed crystals that had been exposed to 456 nm blue light for 15 s, and (Top) 1H NMR of 1T crushed crystals that had been exposed to 456 nm blue light for 15 s and then allowed to thermally relax in the dark for 1 h at RT in accordance with the disclosed subject matter.

FIG. 25 provides a diagram showing an example synthesis of CNAB in accordance with the disclosed subject matter.

FIG. 26 provides a diagram showing an example synthesis of [Cu(CNAB)₄][PF₆] in accordance with the disclosed subject matter.

It is to be understood that both the foregoing general description and the following detailed description are exemplary and are intended to provide further explanation of the disclosed subject matter.

### DETAILED DESCRIPTION

The disclosed subject matter provides photomechanical compounds and methods for producing them. One aspect of the disclosed photomechanical compounds relates to solid-state materials formed of molecular clusters. The disclosed subject matter can be used for a variety of applications such as, for example, and without limitation, electronic materials, including flexible electronic materials such as displays, piezoelectrics, magnetics, semiconductors, photovoltaics, electrically insulating materials, sensors for pressure, gas, temperature, and magnetic fields, coatings, passivation materials, glob top materials, underfill materials, materials for IC, micro-lenses, optical devices, microfluidic devices, microrobots, and the like.

Unless otherwise defined, all technical and scientific terms used herein have the same meaning as commonly understood by one of ordinary skill in the art. In case of conflict, the present document, including definitions, will control.

The terms “comprise(s),” “include(s),” “having,” “has,” “can,” “contain(s),” and variants thereof, as used herein, are intended to be open-ended transitional phrases, terms, or words that do not preclude additional acts or structures. The singular forms “a,” “an,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, e.g., with respect to biological systems or processes, the term can mean within an order of magnitude, within 5-fold, and within 2-fold of a value.

The term “coupled,” as used herein, refers to the connection of a device component to another device component by methods known in the art.

In certain embodiments, the compound can include a solid-state compound having a formula. In non-limiting embodiments, the solid-state compound can include cluster compounds that can be described with the nomenclature for chemical groups. As used herein, for the most part, nomenclature for chemical groups follows the recommendations of “The International Union for Pure and Applied Chemistry”, Principles of Chemical Nomenclature: a Guide to IUPAC Recommendations, Leigh, G. J.; Favre, H. A. and Metanomski, W. V., Blackwell Science, 1998, the disclosure of which is incorporated by reference herein. For example, the solid-state material can be formed from a binary assembly, which can be tunable molecular cluster superatom building blocks. In non-limiting embodiments, the solid-state material can be an organic-inorganic hybrid material. The solid-state material can include a solid-state compound having the formula [Cluster1][Cluster2]. In non-limiting embodiments, the cluster 1 can include copper. For example, the cluster 1 can include a tetrahedral copper (i.e., [Cu(CNAB)₄]. In non-limiting embodiments, the cluster 1 can include azobenzene. For example, the cluster 1 can include an isocyanoazobenzene. In nonlimiting embodiments, the cluster 2 can include a counterion (e.g., [PF₆]).

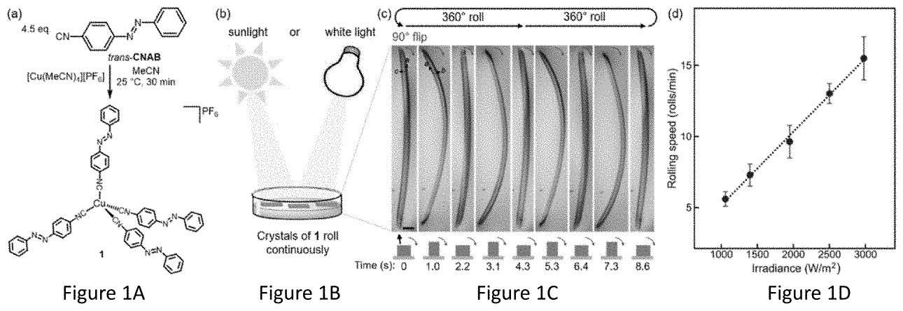

In certain embodiments, the compound can include a formula comprising [Cu(CNAB)₄][PF₆]. In non-limiting embodiments, as shown in FIG. 1A, [Cu(CNAB)₄][PF₆] can be produced by synthesizing 4-isocyanoazobenzene (CNAB) from 4-aminoazobenzene, combining the CNAB with a copper salt in acetonitrile ([Cu(acetonitrile)4][PF6]), and forming a [Cu(CNAB)₄][PF₆] as a powder. In non-limiting embodiments, the compound can be crystalized by diffusing hexanes into a solution of the [Cu(CNAB)₄][PF₆] in tetrahydrofuran (THF).

In certain embodiments, the compound can be in various forms. In non-limiting embodiments, the compound can be in a form of a crystal. For example, the compound can include a rodlike crystal of [Cu(CNAB)₄][PF₆]. In non-limiting embodiments, the crystal can have variable sizes. For example, a ratio of height and length of the crystal can be from about 0.001 to about 0.1, from about 0.001 to about 0.1, from about 0.001 to about 0.1, from about 0.001 to about 0.07, from about 0.001 to about 0.05, from about 0.001 to about 0.03, from about 0.001 to about 0.01, from about 0.005 to about 0.1, from about 0.01 to about 0.1, from about 0.02 to about 0.1, from about 0.03 to about 0.1, from about 0.04 to about 0.1, from about 0.05 to about 0.1, or from about 0.02 to about 0.04. In non-limiting embodiments, a ratio of height and width of the crystal can be from about 0.1 to about 1, from about 0.1 to about 0.9, from about 0.1 to about 0.8, from about 0.1 to about 0.7, from about 0.1 to about 0.6, from about 0.1 to about 0.5, from about 0.2 to about 1, from about 0.3 to about 1, from about 0.4 to about 1, from about 0.5 to about 1, from about 0.6 to about 1, from about 0.7 to about 1, from about 0.8 to about 1, from about 0.9 to about 1, or from about 0.61 to about 0.94.

In certain embodiments, the compound can be in a form of a thin film. The compound in the form of a thin film can be incorporated or coated on a device. The device can include flexible electronic materials (e.g., displays or piezoelectrics), magnetics, semiconductors, photovoltaics, electrically insulating materials, sensors for pressure, gas, temperature, and magnetic fields, coatings, passivation materials, glob top materials, underfill materials, micro-lenses, optical devices, microfluidic devices, microrobots, and the like.

In certain embodiments, the compound can be configured to roll under a light. The light can include sunlight, white light, or blue light. The disclosed compound can be responsive to a broad spectrum (from white light to blue light). In non-limiting embodiments, as shown in FIGS. 1A-1C, the disclosed compound can continuously roll under white light, blue light, or sunlight stimulus. For example, under sunlight, white light, or blue light, the disclosed compound can move by turning over and over on an axis (i.e., rolling) at a predetermined speed without changing the rolling speed or stopping for a predetermined period of time.

In certain embodiments, the rolling speed of the disclosed compound can be adjusted by controlling an irradiance of the light. As shown in FIG. 1D, the rolling speed can be linearly dependent on the light irradiance. For example, the light irradiance can be from about 10 W/m² to about 30000 W/m², from about 10 W/m² to about 20000 W/m², from about 10 W/m² to about 10000 W/m², from about 10 W/m² to about 5000 W/m², from about 10 W/m² to about 4000 W/m², from about 10 W/m² to about 3000 W/m², from about 50 W/m² to about 30000 W/m², from about 100 W/m² to about 30000 W/m², from about 500 W/m²to about 30000 W/m², from about 1000 W/m²to about 30000 W/m², or from about 1000 W/m² to about 3000 W/m². In non-limiting embodiments, the rolling speed can range from about 1 rolls/min to about 1000 rolls/min, from about 1 rolls/min to about 1000 rolls/min, from about 1 rolls/min to about 900 rolls/min, from about 1 rolls/min to about 800 rolls/min, from about 1 rolls/min to about 700 rolls/min, from about 1 rolls/min to about 600 rolls/min, from about 1 rolls/min to about 500 rolls/min, from about 1 rolls/min to about 400 rolls/min, from about 1 rolls/min to about 300 rolls/min, from about 1 rolls/min to about 200 rolls/min, from about 1 rolls/min to about 100 rolls/min, from about 1 rolls/min to about 50 rolls/min, from about 1 rolls/min to about 30 rolls/min, from about 1 rolls/min to about 15 rolls/min, from about 2 rolls/min to about 1000 rolls/min, from about 3 rolls/min to about 1000 rolls/min, from about 4 rolls/min to about 1000 rolls/min, from about 5 rolls/min to about 1000 rolls/min, from about 10 rolls/min to about 1000 rolls/min, from about 15 rolls/min to about 1000 rolls/min, or from about 5 rolls/min to about 15 rolls/min. In non-limiting embodiments, the rolling speed can be at least about 2 rolls/min, at least about 3 rolls/min, at least about 4 rolls/min, or at least about 5 rolls/min. In certain embodiments, the compound (e.g., crystals) can roll faster in a hemispherical cavity (e.g., >60 rolls/min).

In certain embodiments, the disclosed compound can roll upon exposure to light without apparent mechanical fatigue. For example, the compound can be continuously illuminated under white light for at least 1 day (e.g., at least about 100000 full rotations), at least 2 days (e.g., at least about 200000 full rotations), at least 3 days (e.g., at least about 300000 full rotations), at least 4 days (e.g., at least about 400000 full rotations), at least 5 days (e.g., at least about 500000 full rotations), at least 6 days (e.g., at least about 600000 full rotations), at least 7 days (e.g., at least about 700000 full rotations), at least 8 days (e.g., at least about 80000 full rotations), at least 9 days (e.g., at least about 90000 full rotations), or at least 10 days (e.g., at least about 1000000 full rotations) without damage or change in rolling rate.

In certain embodiments, the disclosed compound can include an oil. In non-limiting embodiments, the compound can include an oil as a lubricant. In non-limiting embodiments, the oil can be applied to the compound as a thin coating. With oil as a lubricant, the compound (e.g., crystal) can roll continuously and even move across the slide, while without oil, it can bend or jump.

The disclosed subject matter also provides methods for producing the disclosed compound. An example method can include synthesizing 4-isocyanoazobenzene (CNAB) from 4-aminoazobenzene; combining the CNAB with a copper salt in acetonitrile ([Cu(acetonitrile)4][PF6]); and forming a [Cu(CNAB)₄][PF₆] as a powder. For example, Acetonitrile (12 mL) can be added to a mixture of solid trans-4-isocyanoazobenzene (CNAB) (e.g., about 100 mg, 0.48 mmol) and solid [Cu(CH₃CN)₄][PF₆] (e.g., about 39.8 mg, 0.11 mmol) at room temperature. The reaction mixture can be stirred for about 30 minutes. The reaction can be dried in vacuo and then washed with toluene (e.g., 10 mL) to remove excess CNAB. The remaining solid can be dried to yield [Cu(CNAB)₄][PF₆] (e.g., about 85.7 mg, 0.08 mmol, 77%) as a bright orange solid.

In certain embodiments, the method can further include crystallizing the powder by diffusing hexanes into a solution of the [Cu(CNAB)₄][PF₆] in tetrahydrofuran (THF) to produce at least one crystal. For example, single crystals of the “twisted” form 1T can be grown from a diffusion of hexanes into a concentrated solution of 1 in THF at room temperature. In non-limiting embodiments, crystals of 1T can also be formed from a layering of hexanes over a dilute solution of 1 in THF. Single crystals of the “flat” form 1F can be grown from the cooling of a concentrated solution (e.g., in 1:1 THF:hexanes). After the initial formation of 1F, further crystallization of 1F can be achieved using crushed crystals of 1F as seed crystals. In non-limiting embodiments, the crystal can be a rodlike crystal that can be configured to continuously roll at least about 500,000 rotations without damage.

### EXAMPLES

### Example 1: Highly Twisted Azobenzene Ligand Causes Crystals to Continuously Roll in Sunlight

The disclosed subject matter provides an example of solar-powered continuous motion in azobenzene single crystals. While certain strategies to allow visible light control over azobenzene isomerization can rely on extensive substitution on the azobenzene rings, the disclosed subject matter demonstrates that molecular conformations imposed by crystal packing can produce visible light-responsive azobenzene crystals. The disclosed subject matter shows that a tetrahedral Cu(I)-isocyanoazobenzene complex packs in the solid state with one highly twisted azobenzene ligand, resulting in crystals that continuously roll under white light stimulus (FIGS. 1A-1C), including sunlight.

Combining tetrakis(acetonitrile)copper(I) hexafluorophosphate ([Cu(MeCN)4] [PF6]) with 4-isocyanoazobenzene (CNAB) in acetonitrile at room temperature yields [Cu-(CNAB)4][PF6] as an orange powder (FIG. 1A, marked as 1).

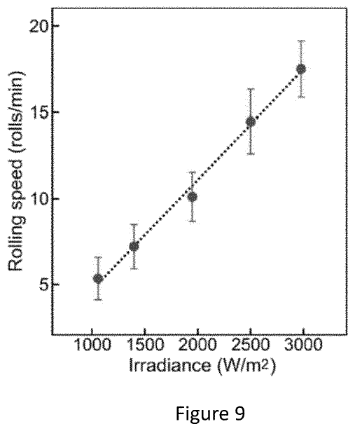

Diffusing hexane vapor into a solution of 1 in tetrahydrofuran (THF) produces rodlike crystals, henceforth 1T, with a variety of dimensions. These crystals are responsive to broad-spectrum white light and blue light. Under white or blue light and a thin coating of oil, thick crystals crack, thin crystals bend, and intermediate-size crystals (height:length about 0.02-0.04, height:width about 0.61-0.94) roll. With oil as a lubricant, the crystals roll continuously and even move across the slide, while without oil, they bend or jump. The rolling speed is linearly dependent on the light irradiance (FIGS. 1D and 9), and the crystals roll faster in a hemispherical cavity (>60 rolls/min). The crystals also roll upon exposure to bright, unfocused sunlight and without apparent mechanical fatigue. One crystal was continuously illuminated under white light for 5 days (500000 full rotations) and observed no damage or change in rolling rate.

Trans-to-cis isomerization of azobenzene molecules occurs at the exposed surface, with the degree of isomerization decreasing with distance from the surface and giving rise to strain from the difference between the lattice of the cis molecules and that of the trans. This strain causes the crystals to crack or bend toward or away from the light source. Limited light penetration into the crystal means that as little as 1% of the azobenzene molecules are isomerized.

1T crystals bend and crack by the mechanism described above. When crushed 1T crystals are dissolved and examined by ¹H NMR, all the CNAB units are trans. After 15 s of exposure to 456 nm blue light, the dissolution of the crushed crystals results in a ¹H NMR with 2.5% of the CNAB units isomerized to the cis isomer. This cis percentage decreases as the crystals thermally relax back to the all-trans state in the dark. 1T crystals bend away from the light source, indicating that the cis lattice is expanded relative to that of the trans, because isomerization leads to expansion of the light-exposed face relative to the dark face of the crystal.

As the light used to roll the crystals also provides heat, the rolling behavior was assessed at various temperatures to further demonstrate that the rolling is due to isomerization rather than a photothermal process. A 1T crystal under white light rolls at 15, 5, and −5° C., while a 1T crystal that rolls at 25° C. stops rolling at 40 and 60° C. Higher temperatures prevent rolling, likely by allowing rapid isomerization back to the more thermally stable trans isomer.

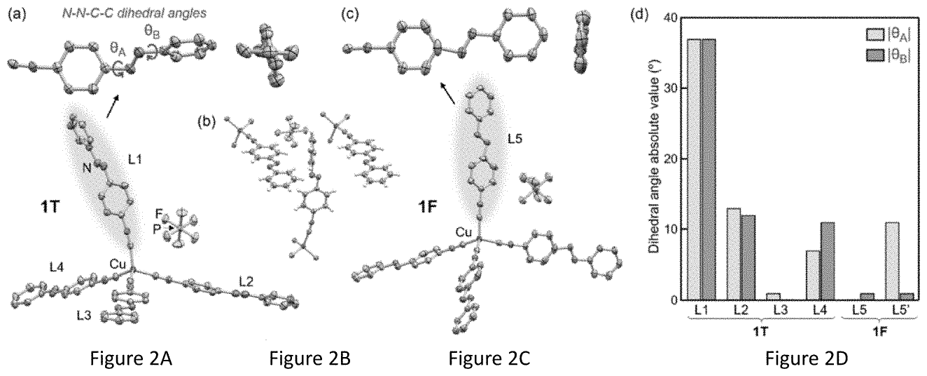

Single-crystal X-ray diffraction (SCXRD) reveals that 1T features a highly twisted azobenzene (FIGS. 2A and 2B). The twist of the phenyl rings with respect to the N═N bond is described by the N═N—C—C dihedral angles, θA and θB (FIG. 2D). In 1T, two azobenzenes are twisted out of planarity, one with θ_{A}=−13° and θ_{B}=−12° and another with θA=θB=37°. In contrast to 1T, crystals of an undistorted polymorph of 1 (1F, FIG. 2C) formed by slow cooling of a saturated solution of 1 in 1:1 hexanes: THF do not respond to blue or white light, suggesting that the photomechanical response of 1T crystals arises from the severely twisted azobenzene.

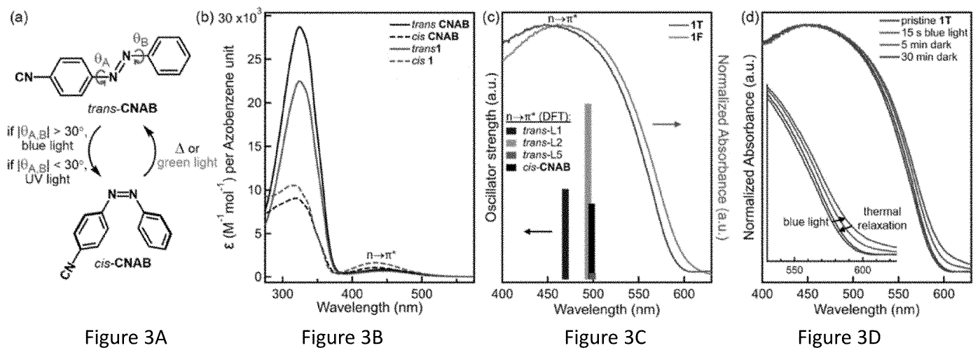

The trans and cis isomers of azobenzene feature overlapping bands in the UV (π-π*) and the visible (n-π*), as do CNAB and 1 in solution (FIGS. 3A and 3B). UV light drives trans-to-cis isomerization, and visible light drives back to the cis isomer, as the trans has the higher extinction coefficient in the UV while that of the cis is higher in the visible. Shifting the trans n-π* band away from the cis peak allows for visible light isomerization in both directions.

The diffuse reflectance electronic absorption spectra of 1T and 1F show that the n-π* transition of 1T is blue-shifted compared to that of 1F (FIG. 3C). Because the spectrum of 1T includes contributions from all four azobenzene units, the blue shift of the most twisted azobenzene is likely even larger than what is observed in the aggregate. The shift is consistent with the disclosed calculations on trans-CNAB (FIG. 3C).

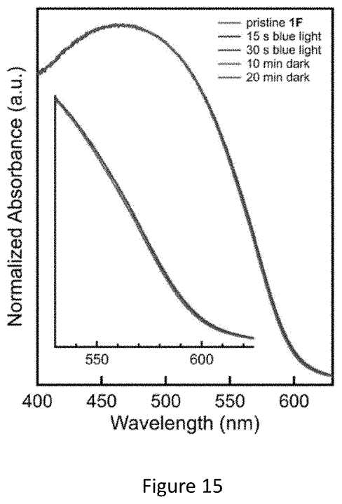

Unlike in ortho-substituted azobenzenes where green light drives trans-to-cis isomerization due to red-shifting of the trans n-π* peak, here crystal packing allows blue light to drive the same process via a twist-induced blue-shift of the trans n-n-π* peak. These techniques were verified by observing the change in the n-π* peak of 1T after irradiation with 456 nm blue light of irradiance 329 W/m²-the n-π* peak is red-shifted by the partial trans-to-cis isomerization in the solid state, reverting to the original signal gradually over 60 min in the dark, consistent with thermal relaxation back to the trans (FIG. 3D). This demonstrates that the trans n-π* transition in 1T is higher energy than that of the cis. By contrast, 1F crystals show no change in absorbance after irradiation with blue light (FIG. 15), consistent with the assignment of the blue-light responsive nature of 1T to its highly twisted CNAB arm. This both accounts for the photomechanical response of 1T crystals to blue and white light and establishes shape-constrained crystal packing as a powerful design strategy for the manipulation of chromophores.

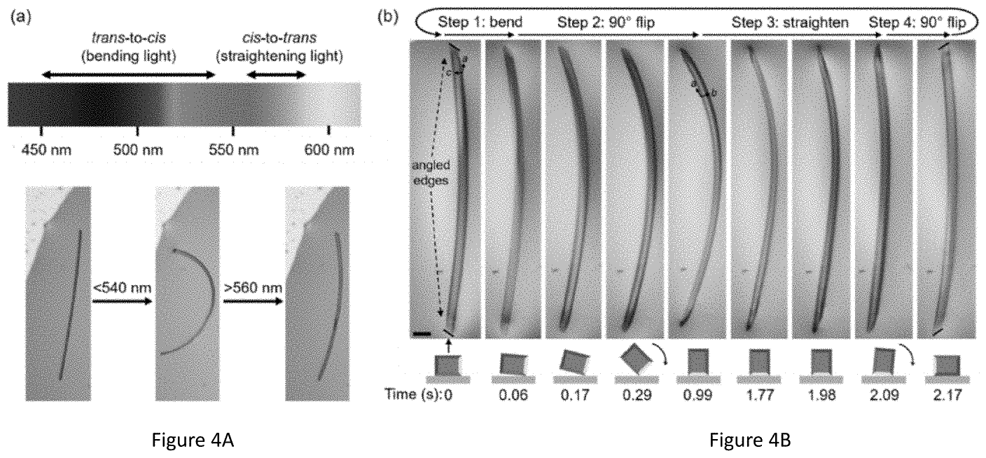

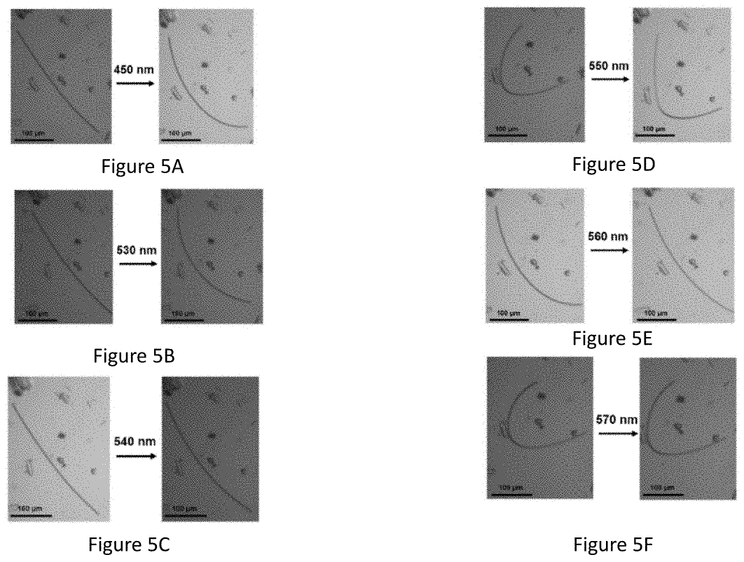

To further isolate the effects of specific wavelengths of light on the rolling process itself, 1T crystals were irradiated using bandpass filters. The bending and straightening of a thin crystal signify surface isomerization to cis and trans, respectively. Light from 450 to 530 nm bends crystals, light from 530 to 560 nm produces no response, and light from 560 to 580 nm straightens crystals (FIGS. 4A and 5). Thus, it was shown that blue light isomerizes azobenzenes at the surface of 1T from trans to cis, while green light reverses the process, consistent with both spectroscopic data and calculations.

The motion of a 1T crystal was parsed into four stages: (1) starting with the widest part of the crystal, the ac face, facing up, the crystal bends away from the light, (2) it tips onto its thinner side such that the ab face is now face up, (3) it straightens, and (4) it is unstable on its thin edge and falls over such that the top face is now the opposite ac face to that which was originally face up (FIG. 4B). The continued rolling is the iteration of this process. The tipping in stage 2 can be attributed to the slanted crystal ends-a feature that also contributes to the rolling mechanism of azobenzene crystals that roll briefly when heated through a thermal phase transition. The rolling mechanism of the 1T crystals is also similar to the thermally induced rolling mechanism of liquid crystalline elastomer rods prepared by the Cai group.

While the bending process can beattributable to blue light, straightening (stage 3) can be due to cis-to-trans isomerization of the bent face by green light, trans-to-cis isomerization of the newly exposed opposite face by blue light, or both. A crystal was irradiated by using only 450-550 nm light, excluding light that drives the cis-to-trans isomerization. Though the crystal still rolled, stage 3 was markedly slower (−11 s) compared to the same crystal under broad-spectrum white light (−1.5 s). While blue light alone is sufficient to roll the crystals, the fast-rolling under white light is actively facilitated by the presence of green light and, thus, by both trans-to-cis and cis-to-trans isomerization.

The disclosed subject matter presents apicture of how and why 1T crystals roll under blue light, white light, and sunlight and demonstrate that crystal packing alone can dramatically change the electronic absorption and physical response of an azobenzene chromophore. Given these results, the electronic manipulation of well-known chromophores by crystal packing using novel geometric templates can be a rich and productive area. The crystals described above exhibit complex and continuous motion and are easily prepared from commercial starting materials, making them excellent candidates for solar-driven micro-actuators.

FIGS. 5A-5G provide optical microscope images showing that (5A-5C) straight crystals bend under (5A) 450 nm and (5B) 530 nm light but not under (5C) 540 nm light, and (5D-5F) bent crystals straighten under (5E) 560 nm light but not under (5D) 550 nm light or (5F) 570 nm light.

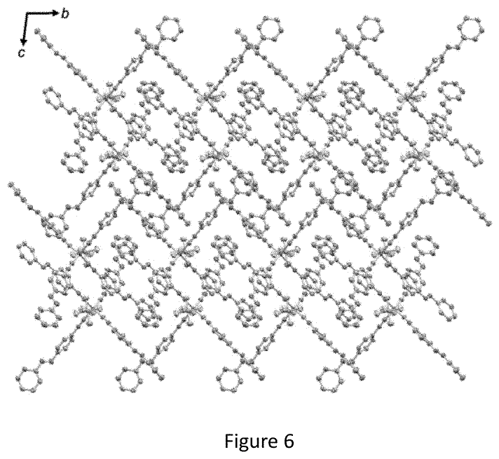

FIG. 6 provides a diagram showing solid-state molecular packing of [Cu(CNAB)4][PF6] (1T) with hydrogens omitted for clarity. Thermal ellipsoids are set at a 50% probability level (Atom color-coding: Cu orange-red, N blue, F yellow-green, P orange, and C grey).

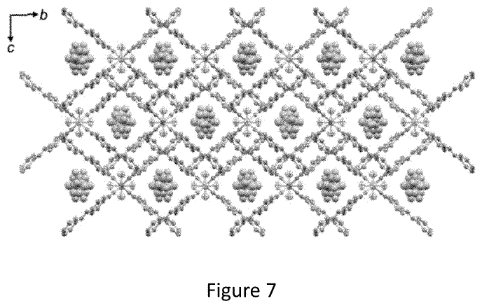

FIG. 7 provides a diagram showing solid-state molecular packing of [Cu(CNAB)4][PF6]⋅hexanes (1F) showing the disordered hexane channels. Only L5 (65% occupancy) is shown, and L5′ (35% occupancy) is omitted for clarity. Thermal ellipsoids are set at a 50% probability level (Atom color-coding: Cu orange-red, N blue, F yellow-green, P orange, and C grey).

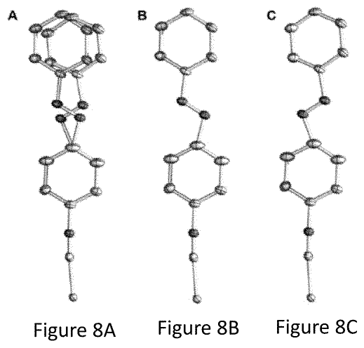

FIGS. 8A-8C provide diagrams showing solid-state molecular structure of the asymmetric unit of [Cu(CNAB)4][PF6]⋅hexanes (1F) showing the disordered CNAB fragment with (8B) L5 65% and (8C) L5′ 35% occupancy. The solvent molecules (hexanes), hexafluorophosphate counterion, and all hydrogen atoms have been omitted for clarity. Thermal ellipsoids are set at a 30% probability level (Atom color-coding: Cu pale copper, N blue, and C grey).

TABLE 1
Rolling rate data for five crystals each from two batches of 1T. The average for
batch 2 is plotted in FIG. 1, and the average for batch 1 is plotted in FIG. 8.

| | Irradiance | Rolls/min | | | | | | |
|---|---|---|---|---|---|---|---|---|
| | (Wm⁻²) | Crystal 1 | Crystal 2 | Crystal 3 | Crystal 4 | Crystal 5 | Average | StdDev |
| Batch 1 | 1057 | 3.08 | 5.35 | 6.63 | 4.87 | 6.80 | 5.35 | 1.23 |
| | 1396 | 4.80 | 8.51 | 7.77 | 6.52 | 8.46 | 7.21 | 1.28 |
| | 1948 | 7.17 | 10.99 | 11.36 | 9.85 | 11.22 | 10.12 | 1.43 |
| | 2500 | 11.25 | 14.93 | 16.15 | 13.12 | 16.89 | 14.47 | 1.87 |
| | 2978 | 15.21 | 18.07 | 19.35 | 15.59 | 19.33 | 17.51 | 1.63 |
| Batch 2 | 1057 | 5.15 | 6.33 | 5.02 | 6.08 | 5.43 | 5.60 | 0.51 |
| | 1396 | 6.76 | 8.61 | 7.20 | 7.57 | 6.34 | 7.29 | 0.78 |
| | 1948 | 8.59 | 11.28 | 10.45 | 9.66 | 8.18 | 9.63 | 1.15 |
| | 2500 | 12.83 | 13.33 | 13.94 | 13.18 | 11.82 | 13.02 | 0.70 |
| | 2978 | 15.15 | 18.18 | 15.27 | 15.43 | 13.48 | 15.50 | 1.51 |

FIG. 9 provides a graph showing the average rate of rolling vs. white light irradiance (measured at 532 nm) for five crystals from Batch 1 of 1T (see Table 2). The error bars represent the standard deviations, and the dashed line is the linear fit (slope=6.4×10-3, y-intercept −1.72).

TABLE 2
Crystallographic data of the disclosed subject matter.

| | [Cu(CNAB)₄][PF₆] | [Cu(CNAB)₄][PF₆]•hexanes |
|---|---|---|
| | (1T) | (1F) |
| Chemical | C₆₂H₃₆CuN₁₂PF₆ | C₆₂H₃₆CuN₁₂PF₆ |
| formula | | |
| Formula weight | 1037.44 | 1157.54 |
| Space group | P1̅ | Fddd |
| a (Å) | 10.547(1) | 13.8436(4) |
| b (Å) | 12.4905(9) | 23.8017(8) |
| c (Å) | 19.204(1) | 36.012(1) |
| α (deg) | 98.737(5) | 90 |
| β (deg) | 93.850(6) | 90 |
| γ (deg) | 100.344(7) | 90 |
| V (Å³) | 2448.2(3) | 11865.9(6) |
| Z | 2 | 8 |
| μ (mm⁻¹) | 1.562 | 1.351 |
| T (K) | 100 | 100 |
| GOF (S) | 1.023 | 1.061 |
| [all data] | | |
| R1^{a} (wR2^{b}) | 0.0708 (0.1719) | 0.0726 (0.2086) |
| [I > 2σ(I)] | | |
| R1^{a} (wR2^{b}) | 0.1074 (0.1990) | 0.0788 (0.2198) |
| [all data] | | |
| Reflections | 8509 | 2855 |
| Radiation type | Cu Kα | Cu Kα |

^{a}R1 = Σ[w(Fₒ − F_{c})]/Σ[wFₙ];
^{b}wR2 = [Σ[w(Fₒ² − F_{c}²)²]/Σ[w(Fₒ²)²]]^{1/2}, w = 1/[σ²(Fₒ²) + (aP)² + bP], where P = [max(Fₒ², 0) + 2(F_{c}²)]/3

Instrumentation: All reactions were performed open to an atmosphere with magnetic stirring unless otherwise noted. All commercial reagents and solvents were used as provided. All final products were dried in vacuo prior to reporting yields. ¹H, ₁₃C, ¹⁹F and ³¹P NMR spectra were recorded on Bruker DRX400 and DMX500 spectrometers in deuterated solvent and at frequencies as noted. Data for ₁H NMR are reported as follows:

chemical shift (6, in ppm), multiplicity (s=singlet, d=doublet, t=triplet, q=quartet, m=multiplet), coupling constant (J, in Hz), and integration (b=broad). Data for ₁₃C, ¹⁹F and ³¹P NMR are reported in terms of chemical shift. Electronic absorption spectra were recorded on an Agilent Cary 5000 UV-Vis-NIR spectrophotometer in a 1.0 cm quartz cell or with an external Diffuse Reflectance Accessory. Infrared (IR) spectra were recorded on a Perkin Elmer Spectrum400 FTIR spectrometer using a PIKE ATR attachment.

Observations of crystal behavior, including cracking, bending, and rolling, were all conducted using aNikon ECLIPSE LV150N optical microscope equipped with aNikon LV-HL50W 12V 50W LONGLIFE halogen lamp. Wavelength filters (bandpass, longpass, and shortpass) were purchased from ThorLabs. A 456 nm Kessil lamp was used as a source of blue light for the blue-light-only experiments listed below.

Tetrakis(acetonitrile)copper(I) hexafluorophosphate ([Cu(MeCN)₄][PF₆]) was prepared according to a literature procedure but can be purchased from Strem Chemicals. All other commercially available compounds were purchased from Sigma-Aldrich, TCI, Alfa-Aesar and Acros. All solvents and reagents were directly used as purchased without any further purification.

X-ray Diffraction Techniques: All structures were collected on an Agilent SuperNova diffractometer using mirror-monochromated Cu Kα radiation. Crystals were cooled to 100 K during collection using an Oxford cryostream cooling device. Crystals were mounted on a MiTeGen MicroMount pin using Paratone N oil. Data collection, integration, scaling (ABSPACK) and absorption correction (face-indexed Gaussian integration) were performed in CrysAlisPro. Space group assignments were determined by examination of systematic absences, E-statistics, and successful refinement of the structures. The structures were solved by intrinsic phasing or direct methods using SHELXS-97 and refined against F² on all data by full matrix least squares with SHELXL-97 with the OLEX 2 interface. All non-hydrogen atoms were refined anisotropically except in the disordered solvent. Hydrogen atoms were placed at idealized positions and refined using a riding model. Further details for individual structures are noted below, and crystallographic data for 1T and 1F are given in Table 2.

[Cu(CNAB)₄][PF₆] (1T). The structure was solved in the triclinic space group P1 with two molecules of 1T per unit cell. The asymmetric unit contains one molecule of 1T. There is no disorder in the structure, nor are there any solvent molecules.

[Cu(CNAB)₄][PF₆]⋅hexanes (1F). The structure was solved in the orthorhombic space group Fddd with two molecules of 1F per unit cell. The asymmetric unit was found to contain one-quarter of 1F, and the rest of the molecule is generated by symmetry. The asymmetric unit also contains roughly one-half of one highly disordered hexane molecule. The highly disordered hexanes in the structure were partly modeled in order to avoid the use of a solvent mask and clearly indicate the presence of a disordered solvent, but complete modeling was not possible due to the extent of the disorder. The carbon atoms of the disordered hexanes are thus modeled isotropically, and hydrogen atoms have not been added to these fragments.

Diffuse Reflectance Solid-State Electronic Absorption Spectra Collection Methodology

Crushed crystals: Crystals of 1T were isolated and briefly dried in vacuo, crushed to a fine powder using a mortar and pestle, and then loaded into a sample holder consisting of a metal plate with a quartz cover sheet. The diffuse reflectance electronic absorption spectra for the crystals were then collected from 350 to 800 nm using an Agilent Cary 5000 UV-Vis-NIR spectrophotometer with an external Diffuse Reflectance Accessory. First, a spectrum was collected of the 1T sample after sitting in the dark for 1 h. The sample was then exposed to 456 nm light with an irradiance of 329 W/m² for 5, 10, and 15 s and spectra were collected after each exposure. As there was no change between 10 s and 15 s, 15 s of exposure time was taken as producing the maximum amount of surface isomerization of the 1T crystals. Spectra were subsequently collected over the following 60 minutes as the sample sat in the dark, thermally relaxing back to the all-trans state. Due to the large volume of hexanes-filled channels in 1F, the comparison between the solid-state absorption spectra of 1T and 1F was made using intact crystals as described below since crushing the crystals of 1F can impact their properties by compacting or completely collapsing these hexanes-filled channels.

Intact crystals: Crystals of 1T or 1F were isolated and briefly dried in vacuo, and then loaded into a sample holder consisting of a metal plate with a quartz cover sheet. The sample holder was tightened sufficiently to secure but not crush the crystals. The diffuse reflectance electronic absorption spectra for the crystals were then collected from 350 to 800 nm using an Agilent Cary 5000 UV-Vis-NIR spectrophotometer with an external Diffuse Reflectance Accessory.

For both 1T and 1F, spectra were first collected on the samples after they sat in the dark for 1 h. Then both samples were exposed to 456 nm light with an irradiance of 329 W/m² for 15 s, and their spectra were collected after this exposure. At this point, the 1F sample was exposed to an additional 15 s of 456 nm light (30 s total exposure) to check that longer exposure still did not result in any isomerization, as evidenced by the electronic absorption spectrum. Spectra of the sample of 1T were subsequently collected over the following 60 minutes as the sample sat in the dark, thermally relaxing back to the all-trans state.

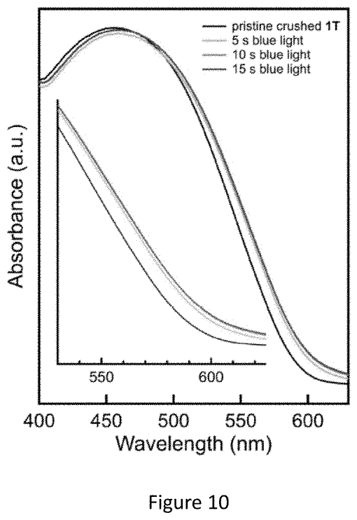

Due to the nature of the diffuse reflectance measurement setup in which the beam path is smaller than the total area of the sample holder, for all spectra enumerated above, a small shift in the orientation or location of the crystals within the sample holder can affect the total absorbance value measured. An effort was made to maintain the distribution of the crystals to make this variation minimal, and both the raw and normalized data are provided in FIGS. 10-15 for transparency.

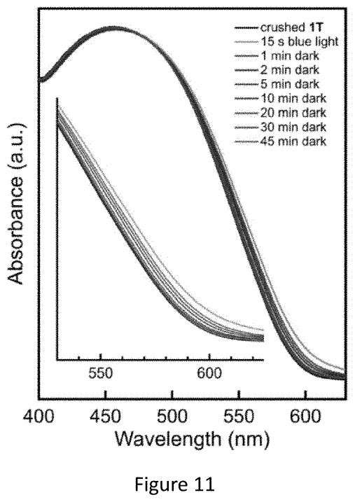

See FIGS. 10 and 11 for results from the crushed crystals of 1T. FIG. 10 provides a graph showing diffuse reflectance solid state electronic absorption spectra of crushed crystals of 1T before and after exposure to 456 nm light of irradiance 329 W/m². FIG. 11 provides a graph showing diffuse reflectance solid state electronic absorption spectra of crushed crystals of 1T before and after exposure to 456 nm light and upon thermal relaxation back to the trans in the dark.

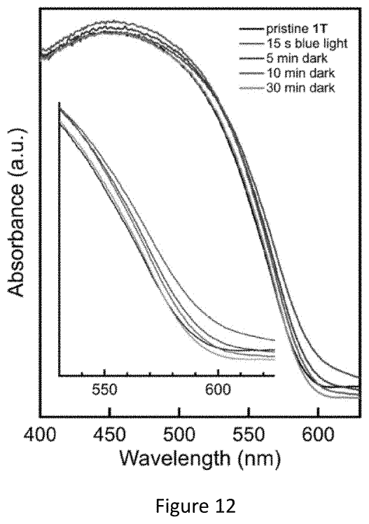

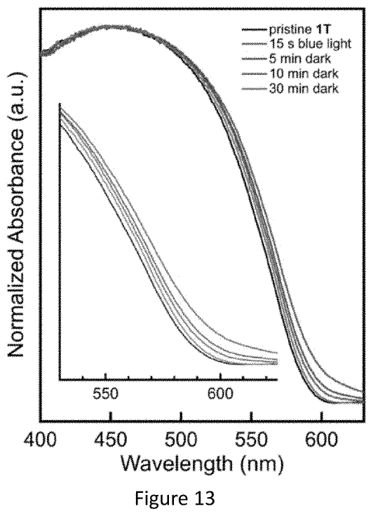

See FIGS. 12 and 13 for results from the intact crystals of 1T. FIG. 12 provides a graph showing diffuse reflectance solid state electronic absorption spectra of intact crystals of 1T before and after exposure to 456 nm light and upon thermal relaxation back to the trans in the dark. FIG. 13 provides a graph showing normalized diffuse reflectance solid state electronic absorption spectra of intact crystals of 1T before and after exposure to 456 nm light and upon thermal relaxation back to the trans in the dark.

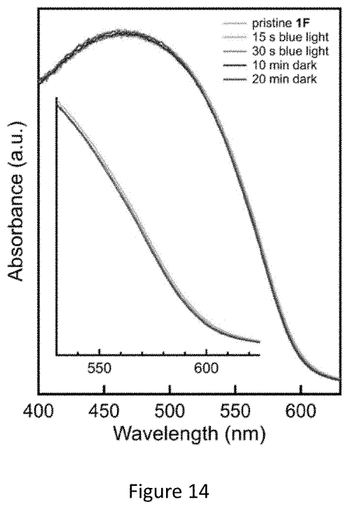

See FIGS. 14 and 15 for results from the intact crystals of 1F. FIG. 14 provides a graph showing diffuse reflectance solid state electronic absorption spectra of intact crystals of 1F before and after exposure to 456 nm light and upon sitting in the dark after the light exposure. FIG. 15 provides a graph showing normalized diffuse reflectance solid state electronic absorption spectra of intact crystals of 1F before and after exposure to 456 nm light and upon sitting in the dark after the light exposure.

Crystal face indexing methodology: Abbreviated collections of three crystals of 1T were obtained on a single crystal X-ray diffractometer using Cu Kα radiation. The faces of these crystals were then indexed as part of the SCXRD data collection. The crystals were then rolled under white light with an irradiance of 3000 W/m² (assessed at 532 nm), and the direction along which all three crystals bent was found to be consistent—i.e., in all three cases, the face-up ac face bent away from the light with the ab face as the “side” of the crystal with respect to this bending face. It is further noted that in all three cases, the crystals were longest along the a direction, followed by the c and b directions, respectively. This means that ac face was consistently wider than the ab face. These consistent results of the indexing of crystals of IT with respect to their rolling behavior were then mapped onto the crystal (FIGS. 1 and 4).

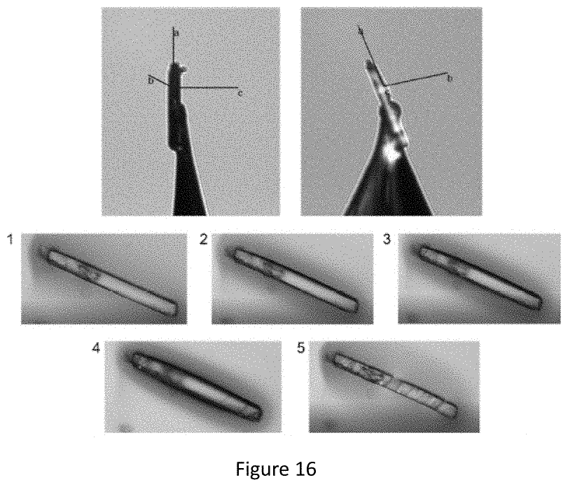

See FIGS. 16-18 for crystal indexing. FIG. 16 provides optical images, including crystallographic axes derived from a partial SCXRD dataset collection on a crystal of 1T (top). Optical microscope images of the same crystal before (1) and during (2-5) exposure to white light (bottom). The crystal first bends away from the light and then cracks. The behavior of the indexed crystal in the light was used to assign the crystallographic directions along which bending and cracking occur.

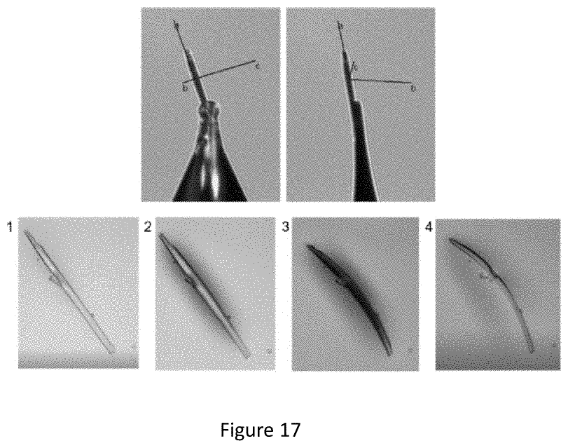

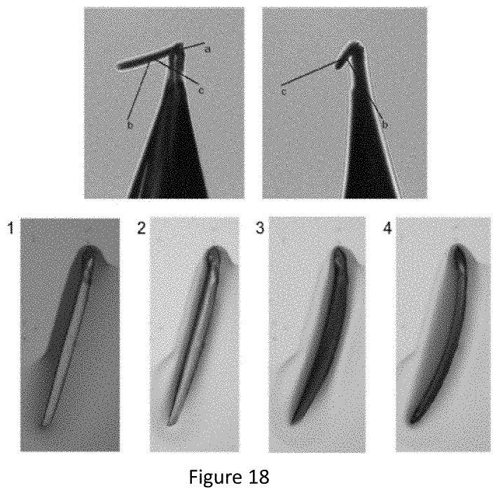

FIG. 17 provides optical images, including crystallographic axes derived from a partial SCXRD dataset collection on a crystal of 1T (top). Optical microscope images of the same crystal before (1) and during (2-4) exposure to white light (bottom). The crystal first bends away from the light and then flips onto its side. The behavior of the indexed crystal in the light was used to assign the crystallographic directions along which bending occurs. FIG. 18 provides optical images, including crystallographic axes derived from a partial SCXRD dataset collection on a crystal of IT (top). Optical microscope images of the same crystal before (1) and during (2-4) exposure to white light (bottom). The crystal first bends away from the light and then flips onto its side. The behavior of the indexed crystal in the light was used to assign the crystallographic directions along which bending occurs.

Observations of Rolling of 1T Crystals and Lack of Light Response of 1F Crystals :

Variable temperature behavior of IT crystals under white light: To better understand the effects of temperature on the rolling behavior of IT crystals, a crystal of 1T was subjected to white light with an irradiance of 3000 W/m² (assessed at 532 nm) at −5, 5, and 15° C. The crystal was observed to roll at all three of these low temperatures, albeit slightly more slowly, as the temperature was lowered. A different crystal of IT was subjected to white light with an irradiance of 3000 W/m² (assessed at 532 nm) at 25, 40, and 60° C. The crystal rolled at 25° C. before and after heating to higher temperatures but did not roll at 40 or 60° C.

Observation of lack of response of 1F to white and blue light: Three crystals of 1F were examined before, during, and after exposure to white light with an irradiance of 3000 W/m² (assessed at 532 nm) for 30 s using the 20× objective on the Nikon optical microscope. The same crystals were also examined before and after 30s exposure to 456 nm light with an irradiance of 329 W/m². In all cases, there was absolutely no change observed in any of the three crystals.

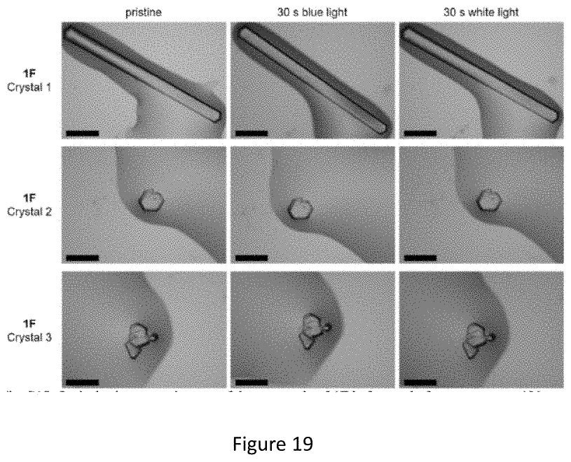

See FIG. 19 for the lack of response of 1F to white and blue light. FIG. 19 shows optical microscope images of three crystals of 1F before and after exposure to 456 nm blue light and white light. The black scale bars represent 100 μm.

Aside from those collected as described below in the sunlight rolling section, all videos were collected using the 20× objective of the Nikon optical microscope, with the addition of filters. Still images were collected on the same microscope using 5× or 20× objectives.

Rate Measurements of Rolling 1T Crystals: The light irradiance was first assessed at five different light intensity settings of the white light on the Nikon optical microscope using the 20× objective. Five crystals, each from two batches of 1T, were then rolled at each of the five light intensity settings under the 20× objective. The rate of rolling was assessed by timing the duration of 5 bending and straightening cycles, constituting 2.5 rolls.

Observation of Sunlight-driven Rolling of 1T Crystals: On a sunny day, crystals of 1T were observed to roll under unfocused sunlight with an irradiance of ˜900 W/m² as assessed at 532 nm. The rolling was observed and recorded using a Nikon SMZ745T microscope.

Rolling crystal endurance test: A small bubble trapped in GE varnish was formed, cured, and then sliced open using a razor blade to form a microscopic dish that can prevent a crystal from rolling out of the field-of-view of the microscope during a multiday rolling experiment. One crystal of 1T was placed inside the dish in minimal Paratone N oil, and this crystal was then rolled under a white light with an irradiance of 3000 W/m² (assessed at 532 nm) and observed using the 20× objective on the Nikon optical microscope. Videos can be used to determine the rolling rate by timing the duration, e.g., of 10 rolls. This rolling rate was then used to determine the total number of rolls completed by the crystal. Due to the impressive speed and endurance of this representative crystal (“Katie Ledecky”), other names more relevant to rolling in pop culture were also considered, such as Rick Astley.

Thermal relaxation of 1T crystal at room temperature: To determine the order of magnitude of thermal relaxation time of intact 1T crystals at room temperature, a rolling crystal of 1T was maximally bent using white light with an irradiance of 3000 W/m² (assessed at 532 nm) and observed using the 20× objective on the Nikon optical microscope as it relaxed at room temperature under ambient light. The crystal was mostly straightened after 10 minutes and fully straightened after 60 minutes.

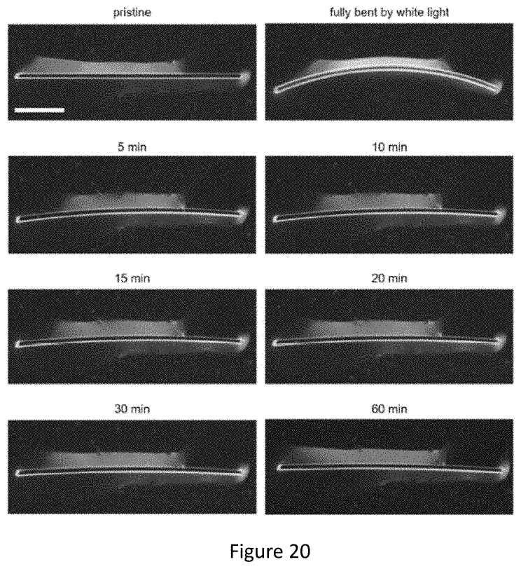

See FIG. 20 for the thermal relaxation of a 1T crystal at room temperature. FIG. 20 shows optical microscope images of the same crystal of 1T before and after exposure to white light and as it relaxed back to the unbent state under ambient light at room temperature. This crystal rolls when continuously exposed to white light. The white scale bar represents 100 μm.

₁H NMR study of 1T isomerization: Given that the solid state electronic absorption spectra of crushed and intact crystals of 1T were found to be equivalent, crushed crystals were used for the ¹H NMR study of 1T isomerization in blue light in order to maximize surface area.

At room temperature in solution, the equilibrium percentage of cis-CNAB in samples of 1 is 16.9%. Equilibration to this point occurred at a maximum rate of 6.53% per hour, starting from dissolved samples of 1T that were 100% trans on dissolution. In order to prevent equilibration in solution from affecting the outcome of the ¹H NMR study of solid state isomerization in 1T, all samples were frozen in liquid nitrogen immediately after dissolution and thawed immediately before their ¹H NMR spectra were collected.

The peak assignments for the trans-CNAB and cis-CNAB arms of 1 used to calculate their relative percentages in solution were made by analogy to the ¹H NMR studies of the trans and cis forms of 4-aminoazobenzene.

A ¹H NMR was first taken of a portion of crushed crystals of 1T that had been sitting in the dark for 12 h. The crushed crystals were then exposed to 456 nm light with an irradiance of 329 W/m² for 15 s, and another portion was dissolved to take the ¹H NMR of this partly isomerized sample. The remaining crushed crystals were allowed to thermally relax back to the trans-state in the dark, and another portion was dissolved to examine the back-isomerization by ¹H NMR.

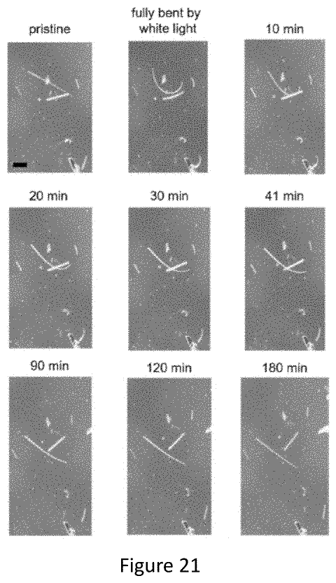

FIG. 21 shows optical microscope images of several crystals of 1T of various dimensions before and after exposure to white light and as they relaxed back to the unbent state under ambient light at room temperature. The black scale bar represents 100 μm.

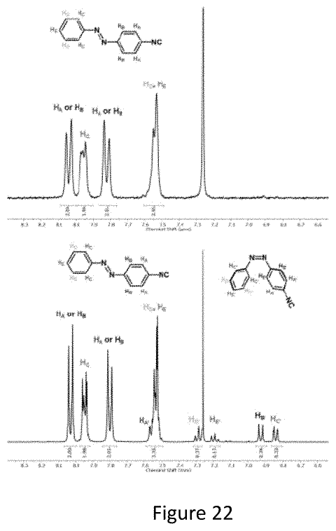

FIG. 22 shows 1H NMR of all-trans-1 obtained by dissolving a sample of 1T crystals, freezing that solution in liquid nitrogen, and then thawing the solution just before a collection of the 1H NMR (top), with tentative peak assignments shown. 1H NMR of a mixture of trans- and cis-1 obtained by irradiating a solution of 1 with UV light for 2 h, again with tentative peak assignments shown. The peak assignments were made by analogy to 4-aminoazobenzene and by examination of coupling constants and peak multiplicity. Regardless of the ambiguity in the assignment of protons HA and HB, the integration of HB′ and HC′ relative to that of the peaks corresponding to HA or HB and HC provides a reliable percentage of isomerization to the cis for a given sample.

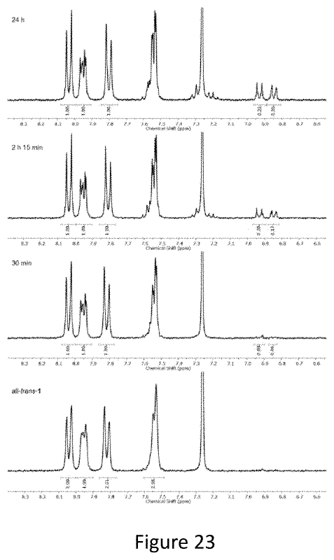

See FIG. 23 for room temperature solution equilibration 1H NMR data for 1. FIG. 23 shows ¹H NMR of all-trans-1 obtained by dissolving a sample of 1T crystals, freezing that solution in liquid nitrogen, and then thawing the solution just before a collection of the ¹H NMR. The solution was then allowed to thermally equilibrate at room temperature in the dark, and 1H NMR spectra were collected at the time intervals shown above. Partial isomerization to cis-1 is evident from the ¹H NMR spectra.

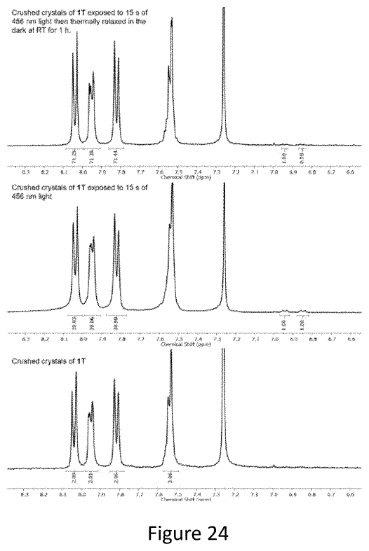

See FIG. 24 for ¹H NMR data on the solid-state isomerization of 1T under blue light. The bottom of FIG. 24 shows ¹H NMR of 1T crushed crystals. The middle of FIG. 24 shows ¹H NMR of 1T crushed crystals that had been exposed to 456 nm blue light for 15 s. The top of FIG. 24 shows ¹H NMR of 1T crushed crystals that had been exposed to 456 nm blue light for 15 s and then allowed to thermally relax in the dark for 1 h at RT. All samples were flash-frozen in liquid nitrogen upon dissolution and thawed immediately before collection of their spectra to avoid confounding effects from thermal equilibration in solution.

TABLE 3
Tabulated results of the integration data presented
in FIG. 24, and the corresponding cis percentages.

| | | | | | H_{A} or | | H_{A} or | | |
|---|---|---|---|---|---|---|---|---|---|
| Sample | | H_{B′} | H_{C′} | cis avg. | H_{B} 1 | H_{C} | H_{B} 2 | trans avg. | % cis |
| 15 | s blue light | 1.00 | 1.00 | 1.00 | 39.03 | 39.06 | 38.98 | 39.02 | 2.5% |
| 1 | h dark | 1.00 | 0.98 | 0.99 | 71.25 | 71.39 | 71.44 | 71.36 | 1.4% |

Computational Methods: Quantum chemical calculations were performed using Jaguar (version9.4, Schrodinger Inc., New York, N.Y., 2016). The geometries for L1, L2, L5, and L5′ were optimized subject to the constraints that the two dihedral angles θ_{A} and θ_{B} were fixed at the values determined from the crystal structure. The geometry of cis-CNAB was fully optimized without constraints. These optimizations applied Density Functional Theory (DFT) using the B3LYP functional and the 631G basis set. Cartesian coordinates for each of the optimized geometries are included in Tables 4-8 below. Using each of these optimized geometries, Time-Dependent DFT (TDDFT) calculations were performed to determine the energies and orbital characterizations of the noted excited states as well as the oscillator strength of each transition. The TDDFT calculations used the larger 6-311G** basis set.

TABLE 4
Dimensions of ten 1T crystals that roll. Height is defined
to be the shortest dimension, width the intermediate
dimension, and length the largest dimension.

| | | | | Height as | Height as |
|---|---|---|---|---|---|
| Height | Width | Length | Volume | % of | % of |
| (μm) | (μm) | (μm) | (μm³) | Length | Width |
| 7.28 | 7.77 | 265.5 | 15017 | 2.74 | 93.75 |
| 6.80 | 7.28 | 159.7 | 7903 | 4.26 | 93.33 |
| 6.31 | 7.28 | 283.5 | 13027 | 2.23 | 86.67 |
| 7.77 | 10.68 | 327.7 | 27180 | 2.37 | 72.73 |
| 8.25 | 11.17 | 298.5 | 27507 | 2.76 | 73.91 |
| 6.31 | 9.71 | 318.4 | 19511 | 1.98 | 65.00 |
| 7.28 | 7.77 | 198.1 | 11201 | 3.68 | 93.75 |
| 5.83 | 7.28 | 242.7 | 10295 | 2.40 | 80.00 |
| 9.22 | 12.14 | 274.8 | 30754 | 3.36 | 76.00 |
| 6.80 | 11.17 | 201.5 | 15286 | 3.37 | 60.87 |

TABLE 5
Geometric coordinates for trans-L1.

| Atom | X | Y | Z |
|---|---|---|---|
| C | 16.28450 | 9.94730 | −1.69680 |
| C | 15.10360 | 9.19220 | −1.71500 |
| C | 13.88120 | 9.83600 | −1.88080 |
| C | 13.83090 | 11.22480 | −2.03030 |
| C | 16.23960 | 11.33310 | −1.89230 |
| C | 23.57080 | 5.11100 | −0.53660 |
| C | 21.33540 | 6.36170 | −0.68370 |
| C | 20.49940 | 6.42810 | 0.43960 |
| C | 19.28260 | 7.09090 | 0.34650 |
| C | 18.91530 | 7.72790 | −0.84530 |
| C | 19.74460 | 7.63850 | −1.97220 |
| C | 20.95270 | 6.95940 | −1.89350 |
| C | 15.01090 | 11.97210 | −2.03340 |
| H | 15.16580 | 8.11550 | −1.60000 |
| H | 12.96400 | 9.25480 | −1.90240 |
| H | 12.87400 | 11.72150 | −2.16160 |
| H | 20.80710 | 5.94880 | 1.36220 |
| H | 18.60710 | 7.13820 | 1.19420 |
| H | 19.42740 | 8.10850 | −2.89630 |
| H | 21.60460 | 6.87650 | −2.75610 |
| H | 14.97280 | 13.04960 | −2.16180 |
| H | 17.17240 | 11.88740 | −1.91670 |
| N | 17.63420 | 8.34240 | −0.87440 |
| N | 17.57630 | 9.36190 | −1.61190 |
| N | 22.54010 | 5.68560 | −0.60410 |

TABLE 6
Geometric coordinates for trans-L2.

| Atom | X | Y | Z |
|---|---|---|---|
| C | 16.26890 | 9.92370 | −1.71880 |
| C | 15.08850 | 9.31690 | −1.25620 |
| C | 13.87680 | 9.98130 | −1.39860 |
| C | 13.82850 | 11.24560 | −1.99820 |
| C | 16.21670 | 11.18130 | −2.33330 |
| C | 23.59360 | 5.14620 | −0.48590 |
| C | 21.34830 | 6.37280 | −0.67440 |
| C | 20.31280 | 6.06610 | 0.21770 |
| C | 19.09760 | 6.72880 | 0.10270 |
| C | 18.91170 | 7.70730 | −0.88170 |
| C | 19.95250 | 8.00370 | −1.77820 |
| C | 21.16530 | 7.34100 | −1.67590 |
| C | 14.99930 | 11.84460 | −2.46390 |
| H | 15.14720 | 8.33670 | −0.79690 |
| H | 12.96090 | 9.51460 | −1.04760 |
| H | 12.87640 | 11.75680 | −2.10710 |
| H | 20.47140 | 5.31230 | 0.98060 |
| H | 18.27400 | 6.50740 | 0.77340 |
| H | 19.78670 | 8.75370 | −2.54250 |
| H | 21.97870 | 7.55300 | −2.36130 |
| H | 14.96200 | 12.82260 | −2.93370 |
| H | 17.14270 | 11.61800 | −2.69350 |
| N | 17.62870 | 8.30920 | −0.91660 |
| N | 17.55460 | 9.33290 | −1.64960 |
| N | 22.55900 | 5.71130 | −0.57420 |

TABLE 7
Geometric coordinates for trans-L5.

| Atom | X | Y | Z |
|---|---|---|---|
| C | 16.25940 | 9.90730 | −1.73420 |
| C | 15.11760 | 9.42220 | −1.07330 |
| C | 13.91290 | 10.10050 | −1.20360 |
| C | 13.83270 | 11.25900 | −1.98640 |
| C | 16.17650 | 11.06310 | −2.52120 |
| C | 23.60350 | 5.16320 | −0.46880 |
| C | 21.35270 | 6.37800 | −0.66920 |
| C | 20.25120 | 5.92440 | 0.06700 |
| C | 19.03610 | 6.58860 | −0.04850 |
| C | 18.90970 | 7.70110 | −0.88990 |
| C | 20.02050 | 8.14890 | −1.62600 |
| C | 21.23540 | 7.49230 | −1.51700 |
| C | 14.96520 | 11.73920 | −2.64440 |
| H | 15.20110 | 8.52390 | −0.47260 |
| H | 13.02710 | 9.72960 | −0.69610 |
| H | 12.88590 | 11.78270 | −2.08220 |
| H | 20.36080 | 5.06210 | 0.71500 |
| H | 18.16470 | 6.26150 | 0.50900 |
| H | 19.90560 | 9.01020 | −2.27340 |
| H | 22.10280 | 7.82320 | −2.07770 |
| H | 14.90370 | 12.63650 | −3.25260 |
| H | 17.07450 | 11.40980 | −3.02260 |
| N | 17.62390 | 8.29790 | −0.92570 |
| N | 17.53870 | 9.30250 | −1.68330 |
| N | 22.56690 | 5.72390 | −0.56200 |

TABLE 8
Geometric coordinates for trans-L5.

| Atom | X | Y | Z |
|---|---|---|---|
| C | 16.26480 | 9.92080 | −1.71610 |
| C | 15.10450 | 9.38580 | −1.13000 |
| C | 13.89550 | 10.05350 | −1.27500 |
| C | 13.82910 | 11.25070 | −1.99850 |
| C | 16.19640 | 11.11700 | −2.44130 |
| C | 23.60610 | 5.17030 | −0.45930 |
| C | 21.35280 | 6.37830 | −0.66980 |
| C | 20.27350 | 5.97690 | 0.12740 |
| C | 19.05480 | 6.63170 | 0.00170 |
| C | 18.90650 | 7.69210 | −0.90140 |
| C | 19.99290 | 8.08430 | −1.70240 |
| C | 21.20980 | 7.43190 | −1.58800 |
| C | 14.98030 | 11.78140 | −2.58090 |
| H | 15.17700 | 8.45800 | −0.57430 |
| H | 12.99550 | 9.64410 | −0.82530 |
| H | 12.87870 | 11.76550 | −2.10690 |
| H | 20.40230 | 5.15900 | 0.82740 |
| H | 18.19840 | 6.33970 | 0.60040 |
| H | 19.85790 | 8.89940 | −2.40330 |
| H | 22.05820 | 7.71850 | −2.19980 |
| H | 14.92980 | 12.70930 | −3.14250 |
| H | 17.10920 | 11.50400 | −2.88300 |
| N | 17.61670 | 8.27810 | −0.95300 |
| N | 17.55150 | 9.33320 | −1.64100 |
| N | 22.56820 | 5.72740 | −0.55760 |

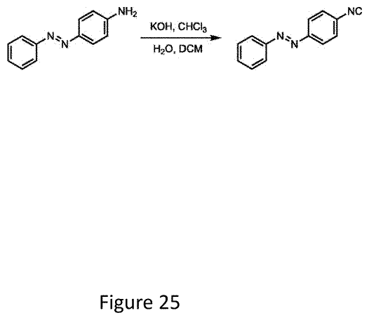

Synthesis and Characterization: As shown in FIG. 25, CNAB was synthesized. A 100 mL round bottom flask equipped with a Teflon-coated stir bar and open to the atmosphere was charged with 4-aminoazobenzene (394 mg, 2 mmol) and DCM (10 mL). A solution of 50% w/w NaOH (5 g in 5 mL) was added, resulting in a biphasic solution. Benzyltriethylammonium chloride (22 mg, 0.1 mmol) was added, and the organic phase immediately turned dark red. Chloroform (1 mL) was added, and the reaction turned hot and slowly turned to a dark brown as it was stirred overnight. After 16 h, the solution was extracted with additional DCM (100 mL), washed with water (50 mL) and brine (50 mL) and dried with Na₂SO₄. The solvent was removed via rotary evaporation, and the residue was purified using basified silica gel flash column chromatography, eluting with DCM/hexanes to afford X as an orange powder. Yield 311 mg, 75%.

¹H NMR (500 MHz, CDCl₃) δ 7.99-7.82 (m, 4H), 7.58-7.50 (m, 5H). IR: 2120 cm⁻¹ (NC).

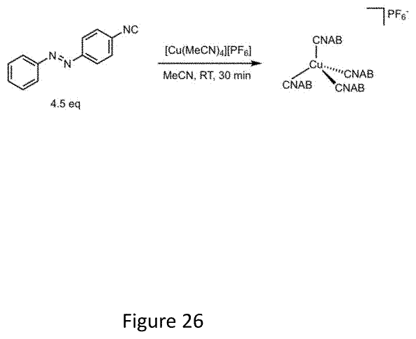

As shown in FIG. 26, Cu(CNAB)₄][PF₆] was prepared. Acetonitrile (12 mL) was added to a mixture of solid trans-4-isocyanoazobenzene (CNAB) (100 mg, 0.48 mmol, 4.5 eq) and solid [Cu(CH₃CN)₄][PF₆] (39.8 mg, 0.11 mmol, 1 eq) at room temperature. The reaction mixture was stirred for 30 minutes. The reaction was dried in vacuo and then washed with toluene (10 mL) to remove excess CNAB. The remaining solid was dried to yield [Cu(CNAB)₄][PF₆] (85.7 mg, 0.08 mmol, 77%) as a bright orange solid. Single crystals of the “twisted” form 1T were grown from the slow diffusion of hexanes into a concentrated solution of 1 in THF at room temperature. Crystals of 1T were also formed from a layering of hexanes over a dilute solution of 1 in THF. Single crystals of the “flat” form 1F were grown from slow cooling of a concentrated solution of 1 in 1:1 THF:hexanes. After the initial formation of 1F further crystallization of 1F was most easily achieved using crushed crystals of 1F as seed crystals: ₁H NMR (500 MHz, CDCl₃) δ 8.04 (d, J=8.7 Hz, 2H), 7.94 (dd, J=7.7, 2.1 Hz, 2H), 7.82 (d, J=8.7 Hz, 2H), 7.58-7.49 (m, 3H). ¹³C NMR (126 MHz, CDCl₃) δ 152.88, 152.35, 132.05, 129.26, 128.01, 124.17, 123.29. ³¹P NMR (202 MHz, CDCl₃) δ 117.61-−161.04 (m). ¹⁹F NMR (471 MHz, CDCl₃) δ −71.95 (d, J=712.6 Hz).

Given that large crystals of 1T crack under blue or white light stimulus rather than bending or rolling, thin crystals of 1T are well-suited to bending, curling, and rolling. A dilute solution of 1 in THF was prepared (˜30-50 mg in 10 mL) and filtered through a pipette filter composed of a glass pipette packed with one half of a glass fiber filter below one inch of Celite. This solution was loaded into the bottom half of a 20 ml vial, and 10 mL of hexanes was carefully layered on top of the THF solution of 1. This crystallization setup was then stored undisturbed in the dark at room temperature for one week.

After one week, the bottom of the vial was covered with long, thin crystals of 1T. From two random samples of 150-200 crystals from recrystallization set up according to the above procedure, ˜8-20% were of the correct dimensions to undergo rolling. Many of the crystals formed small clusters, making individual counting of the total number of crystals challenging, but in the two small sub-samples from the recrystallization, there were 16 and 23 rolling crystals, respectively.

All of the crystals formed can be responsive to white light and blue light, though many will be of appropriate aspect ratios to bend, curl, or crack rather than roll. Because the rolling behavior requires a particular aspect ratio range, the rate of nucleation and growth of the crystallization strongly affects the number of rolling crystals formed. Given that 1 can be prepared inexpensively and easily on a large scale, several individual recrystallizations can be prepared at once such that a good number of rolling crystals can be obtained, accounting for potential variation in nucleation and growth rates between the setups.

If rolling crystals are not reliably obtained using the above methodology, an alternative recrystallization setup can be used wherein a more concentrated solution of 1 in THF (˜20 mg in 3 mL) was filtered through a pipette filter composed of a glass pipette packed with one half of a glass fiber filter below one inch of Celite and transferred into a one-dram vial. The one-dram vial was then put into a five-dram vial, and ˜5 mL of hexanes was added to the five-dram vial. The five-dram vial is then capped, and the setup is left in the dark overnight at room temperature. This yields a more rapid crystallization than the above layering setup, which can provide large numbers of 1T crystals with dimensions appropriate for rolling.

TABLE 9
Geometric coordinates for cis-CNAB.

| Atom | X | Y | Z |
|---|---|---|---|
| C | −4.42480 | −1.25050 | −1.68290 |
| C | −3.11670 | −0.92070 | −2.06760 |
| C | −2.71890 | −1.09500 | −3.39980 |
| C | −3.63120 | −1.52170 | −4.35580 |
| C | −4.94230 | −1.83180 | −3.96990 |
| C | −5.33180 | −1.71130 | −2.62870 |
| N | −2.08230 | −0.60270 | −1.12580 |
| N | −2.19400 | 0.25370 | −0.22330 |
| C | −3.29100 | 1.17320 | −0.12630 |
| C | −3.75230 | 1.93890 | −1.20650 |
| C | −4.72900 | 2.90820 | −0.99270 |
| C | −5.26100 | 3.10760 | 0.28300 |
| C | −4.78660 | 2.35570 | 1.36000 |
| C | −3.78230 | 1.41200 | 1.16350 |
| N | −5.85210 | −2.27360 | −4.91380 |
| C | −6.63410 | −2.64850 | −5.71690 |
| H | −3.33670 | 1.78730 | −2.19650 |
| H | −5.07810 | 3.50950 | −1.82690 |
| H | −6.02990 | 3.85810 | 0.43990 |
| H | −5.18510 | 2.51940 | 2.35680 |
| H | −3.37120 | 0.84490 | 1.99280 |
| H | −1.68930 | −0.88550 | −3.67100 |
| H | −3.33970 | −1.63410 | −5.39420 |
| H | −6.34380 | −1.97650 | −2.34330 |
| H | −4.72600 | −1.15280 | −0.64620 |

TABLE 10
Summary of calculated excitation energies.

| | | | n→π* | Oscillator |
|---|---|---|---|---|
| Ligand | θ_{A} | θ_{B} | (nm) | strength |
| L5 | −0.5 | −3.6 | 496 | 0.000 |
| L5′ | −10.2 | −2.3 | 494 | 0.003 |
| L2 | −13.4 | −12.5 | 491 | 0.120 |
| L1 | −37.2 | −36.7 | 466 | 0.062 |
| cis-CNAB | | | 495 | 0.052 |

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof Those skilled in the art will recognize or be able to ascertain, using no more than routine experimentation, many equivalents to the specific embodiments described herein. Such equivalents are intended to be encompassed by the following claims.

What is claimed is:

1. A compound, comprising
a solid-state compound having a formula including [Cluster1][Cluster2], wherein the Cluster 1 comprises copper and azobenzene, and Cluster 2 comprises a counterion.

2. The compound of claim 1, wherein the cluster 1 comprises tetrahedral copper.

3. The compound of claim 1, wherein the cluster 1 comprises isocyanoazobenzene.

4. The compound of claim 1, wherein the formula comprises [Cu(CNAB)₄][PF₆].

5. The compound of claim 1, wherein the compound is configured to roll under a light.

6. The compound of claim 5, wherein the light comprises a white light, a sunlight, a blue light, or combinations thereof.

7. The compound of claim 1, wherein the compound is in a form of a crystal.

8. The compound of claim 7, wherein a ratio of a height and a length of the crystal ranges from about 0.02 to about 0.04.

9. The compound of claim 7, wherein a ratio of height and width of the crystal is from about 0.61 to about 0.94.

10. The compound of claim 7, wherein the crystal is configured to continuously roll at least about 500,000 rotations without damage.

11. The compound of claim 1, wherein a rolling speed of the compound is adjustable based on an irradiance of the light.

12. The compound of claim 1, wherein the compound comprises an oil.

13. The compound of claim 1, wherein the compound is in a form of a thin film.

14. The compound of claim 13, wherein the thin film is configured to be incorporated into a device.

15. The compound of claim 14, wherein the device can include a microrobot or a microfluid device.

16. A method for producing a photomechanical material, comprising:
synthesizing 4-isocyanoazobenzene (CNAB) from 4-aminoazobenzene;
combining the CNAB with a copper salt in acetonitrile ([Cu(acetonitrile)4] [PF6]); and
forming a [Cu(CNAB)₄][PF₆] as a powder.

17. The method of claim 16, further comprising crystallizing the powder by diffusing hexanes into a solution of the [Cu(CNAB)₄][PF₆] in tetrahydrofuran (THF) to produce at least one crystal.

18. The method of claim 17, wherein the crystal is a rodlike crystal.

19. The method of claim 17, wherein a ratio of height and length of the crystal is from about 0.02 to about 0.04, and a ratio of height and width of the crystal is from about 0.61 to about 0.94.

20. The method of claim 17, wherein the crystal is configured to roll at least about 500,000 rotations without damage.
